(19) United States
(12) Patent Application Publication
(10) Pub. No.: US 2021/0085329 A1
(43) Pub. Date: Mar. 25, 2021

(54) LAPAROSCOPIC DEVICE FOR LOCATING, MOBILISING AND CLAMPING ORGANS OR TUBULAR STRUCTURES

(71) Applicant: Francesco GRAVINA, Rimini (IT)

(72) Inventor: Francesco GRAVINA, Rimini (IT)

(21) Appl. No.: 16/976,091

(22) PCT Filed: Feb. 21, 2019
(86) PCT No.: PCT/IT2019/050034

§ 371 Date: Aug. 27, 2020

(30) Foreign Application Priority Data
Feb. 27, 2018 (IT) 102018000003059

(51) Int. Cl. A61B 17/12

(52) U.S. Cl. CPC A61B 17/12013; A61B 2017/12018

(57) ABSTRACT

The present invention relates to a laparoscopic device (1) for locating, mobilising and clamping organs or tubular structures comprising: a hollow introducer element (2), provided with a first end (21) and a second end (22); an elastic element (3) insertable in said introducer element (2) through said first (21) and second end (22) folded upon itself; a fixing element (4), couplable to said introducer element (2), able to block said elastic element (3) in a predetermined position.

The present invention relates to a laparoscopic device for locating, mobilising and clamping organs or tubular structures.

In greater detail the invention relates to a device of the said type, designed and realised in particular for carrying out closures, i.e. clamping, of the hepatoduodenal ligament, known as Pringle manoeuvres, during laparoscopic operations for hepatic resections, but which can be used for any resection of organs having a tubular structure such as vessels, viscera, ureters and the like, where a reversible closure is necessary during a laparoscopic operation.

The following description relates to a carrying out of Pringle manoeuvres in laparoscopy, but it is very apparent that this should not be considered limited to this specific use.

As is known, laparoscopy is a surgical technique which comprises carrying out a surgical operation by making small incisions, known as mini-laparotomies, of length of 5 or 10 or 12 mm, for insertion of trocar tubes, through which gases such as CO₂ are introduced into the body cavities, as well as optical instruments for the tv camera and appropriate instruments for performing the operation, known as laparoscopic instruments.

Each surgical operation is carried out with the intent to avoid blood loss as far as possible, by cauterising, application of clips, ligatures using threads, and like haemostasis procedures.

Sometimes it is necessary to temporarily block blood circulation in order to enable the operator to carry out the appropriate operation; this temporary interruption is carried out using angiostatins or by use of loops, which are positioned about the vessel and subsequently placed in traction, clamping the vessel to interrupt the passage of blood, and then loosened and removed to reactivate the blood circulation.

This manoeuvre is reversible and repeatable, and is appropriately utilisable not only in operations on vessels in vascular surgery, but also in organ resections and more specifically hepatic resections where the ability of the liver to tolerate the ischemia enables the operator to manage and control haemostasis, employing the Pringle manoeuvre.

The reversible and repetitive nature of this manoeuvre is fundamental for enabling the return to blood circulation without which the organ would suffer irreversible damage.

To today, though there exist laparotomy loops, used in open surgery, i.e. with an opening of the body wall; laparoscopic loops are not known.

In fact, at present the operators performing laparoscopic operations utilise devices used for other aims, which are not completely safe for the patient.

In particular, the ligatures currently used are made of a non-elastic material, so that when utilised in the Pringle manoeuvre they exert a traction on the hepatoduodenal ligament with an increase of the risk of laceration by tearing of the ligament.

Further, the ligatures are knotted to one another, often causing problems of stability and knot tightness.

For the purpose of carrying out a laparoscopy operation, suitable operating instrumentation is required.

In light of the above, the aim of the present invention is therefore to provide a laparoscopic device that is safe and simple to use and which is specific for locating, mobilising and clamping tubular organs.

The present invention therefore specifically relates to a laparoscopic device for locating, mobilising and clamping organs or tubular structures, comprising a hollow introducer element, provided with a first end and a second end, an elastic element suitable for inserting in said introducer element through said first and second end folded on itself so as to form a loop for enveloping said organs or tubular structures, a fixing element, couplable to said introducer element, able to block said elastic element in a predetermined position.

Further, according to the invention, said elastic element is provided with a first end and a second end insertable through said first end of said introducer element so as to exit and re-enter through said second end of said introducer element to form a loop.

According to the invention, said introducer element is preferably provided with a main opening located at said first end at which a deformable element is coupled.

Further, according to the invention, said device can comprise a protection element couplable to said second end, and a second opening is afforded on said protection element for passage of said second end of said elastic element.

Further according to the invention, said fixing element comprises a first end and a second end, both provided with through-holes for enabling passage of said elastic element.

According to the invention said second end preferably has an appendage able to couple with said deformable element of said introducer element.

Again, according to the invention, a first slot and a second slot, diametrically opposite said first slot, are afforded on the external surface of said fixing element. Further, according to the invention, said first slot is provided with an upper end and a lower end, and said second slot is provided with an upper end and a lower end.

According to the invention, said device can preferably comprise a blocking and unblocking organ able to engage with said first slot and second slot, for blocking said elastic element in a predefined position.

Once more according to the invention, said blocking and unblocking organ comprises a substantially spherical organ connected to a first slider and to a second slider, positioned diametrically opposite one another with respect to said spherical organ.

The invention is now described, by way of example and without limiting the scope of the invention, with reference to the accompanying drawings which illustrate preferred embodiments thereof, in which:

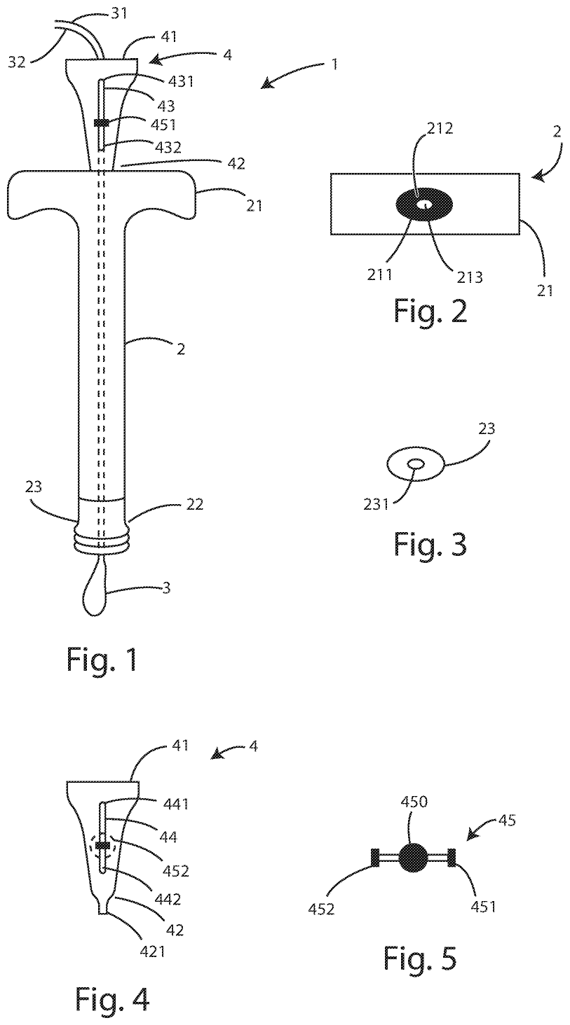

FIG. 1 shows a schematic front view of the laparoscopic device for locating, mobilising and clamping tubular organs, object of the present invention;

FIG. 2 shows a schematic view from above of a portion of the device of FIG. 1;

FIG. 3 shows a schematic view from below of a part of the device of FIG. 1;

FIG. 4 shows a schematic front view of a part of the device of FIG. 1; and

FIG. 5 shows a schematic view from above of a part of FIG. 4.

The similar parts will be indicated in the various drawings with the same numerical references.

With reference to the accompanying figures, the laparoscopic device 1 for locating, mobilising and clamping tubular organs, object of the present invention, essentially comprises an introducer element 2 internally of which an elastic or ligature 3 is inserted, and a fixing element 4 of said ligature 3.

Said introducer element 2 is an internally hollow elongate tubular body made of a stiff transparent material, preferably having a length comprised between 35 and 50 cm and a diameter of 10 mm.

Said introducer element 2 is provided with a first end 21 and a second end 22.

The first end 21 has a preferably rounded conformation so as to enable the operator to have a comfortable grip for manipulation during the Pringle manoeuvre.

A main opening 211 is afforded on the upper face of said first end 21, to which is coupled a deformable element 212.

A first opening 213 is afforded internally of the deformable element 212, to enable passage of said ligature 3 and of the laparoscopic grippers required for the manoeuvre.

A protection element 23 is coupled to the second end 22 which covers said second end 22 and which is made to adhere to the organ during the manoeuvre.

Said protection element 23 is made of a deformable material, preferably silicone clad and transparent, and has a second opening 231 for the passage of said ligature 3.

Said introducer element 2 is suitable to be inserted in tubes or trocars of known type for carrying out the manoeuvre.

Said ligature 3 is a ribbon or a slim tube with a first end 31 and a second end 32.

Said ligature 3 is made of a deformation material, preferably silicone-clad, having a diameter comprised between 8 and 10 French (Fr) and a length comprised between 120 and 160 cm.

Said ligature 3 is radio-opaque, so as to be identified with the use of radioscopic machinery, if necessary during the manoeuvre.

Said second end 32 of said ligature 3 is inserted by means of a laparoscopic gripper internally of said introducer element 2 through said first opening 213 up to exiting from said second opening 231, then to return in reverse so as to form a loop and newly exit from said first opening 213.

Said fixing element 4 has a substantially cylindrical hollow shape, preferably having a length of 5-10 cm.

Said fixing element 4 comprises a first end 41 and a second end 42, both provided with through-holes for enabling the passage of said ligature 3.

Said second end 42 has an appendage 421 able to couple with said deformable element 212 and to engage in said first opening 213.

A first slot 43 and a second slot 44, diametrically opposite to said first slot 43, preferably having a length of 4-6 cm, are afforded on the external surface of said fixing element 4.

Said second slot 43 is provided with an upper end 431, or travel start, and a lower end 432, or travel end.

Likewise, said second slot 44 is provided with an upper end 441, or travel start, and a lower end 442, or travel end.

A blocking and unblocking organ 45 of said ligature 3 is internal of said fixing element 4.

In particular, said blocking and unblocking organ 45 comprises a substantially spherical organ 450 connected to a first slider 451 and to a second slider 452, positioned diametrically opposite one another with respect to said spherical organ 450.

Said spherical organ 450 is preferably made of a stiff material and has a knurled surface and a diameter of 13-15 mm.

Said blocking and unblocking organ 45 engages with said first slot 43 and second slot 44, in particular, said first slider 451 engages with said first slot 43, while said second slider 452 engages with said second slot 44.

Said blocking and unblocking organ 45 passes from a rest or unblocked position, wherein said first slider 451 is arranged in contact with said upper end 431 of said first slot 43 and said second slider 452 is arranged in contact with said upper end 441 of said second slot 44, at a working or blocked position, wherein said first slider 451 is arranged in contact with said lower end 432 of said first slot 43 and said second slider 452 is arranged in contact with said lower end 442 of said second slot 44.

The operation of the laparoscopic device 1 for locating, mobilising and clamping organs or tubular structures, object of the present invention as described in the foregoing, is as follows.

Said introducer element 2 is inserted in a known-type trocar.

Said second end 32 of said ligature 3, as already described, is inserted into said introducer element 2 by means of a first laparoscopic gripper, is passed about the tubular structure of interest on which the Pringle manoeuvre is to be carried out, is gripped by a second laparoscopic gripper to be conducted newly internally of said introducer element 2 through said second opening 231, then to exit said introducer element 2 through said first opening 213.

At the same time, said deformable element 212 nears to contact said tubular structure of interest.

Subsequently, said first 31 and second end 32 of said ligature 3 are inserted in said fixing element 4 first through said appendage 421 and are then conducted outside through said first end 41.

Said fixing element 4 is coupled to said introducer element 2, by inserting said appendage 421 in said first opening 213.

Subsequently, said ligature 3 is set in traction by the operator in a position necessary for carrying out the Pringle manoeuvre and is then blocked, bringing said blocking and unblocking organ 45 from said rest or unblocked position to said working or blocked position, acting on said first slider 451 and second slider 452, as described in the foregoing.

On completion of the Pringle manoeuvre, said ligature 3 is released, unblocking said blocking and unblocking organ 45, i.e. returning said blocking and unblocking organ 45 into said rest or unblocked position.

As is clear from the foregoing description, said device 1 enables carrying out the Pringle manoeuvre in a simple and safe way, preventing damage to the structures involved.

The present invention is described by way of example only, without limiting the scope of application, according to its preferred embodiments, but it shall be understood that the invention may be modified and/or adapted by experts in the field without thereby departing from the scope of the inventive concept, as defined in the attached claims.

1. Laparoscopic device (1) for locating, mobilising and clamping organs or tubular structures comprising:
a hollow introducer element (2), provided with a first end (21) and a second end (22);
an elastic element (3) being insertable in said introducer element (2) through said first (21) and second end (22), folded upon itself so as to form a loop for enveloping said organs or tubular structures;
a fixing element (4), couplable to said introducer element (2), able to block said elastic element (3) in a predetermined position.

2. Device (1) of claim 1 characterised in that said elastic element (3) is provided with a first end (31) and a second end (32) insertable through said first end (21) of said introducer element (2) so as to exit and re-enter through said second end (22) of said introducer element (2) to form a loop.

3. Device (1) according to claim 1, characterised in that said introducer element (2) is provided with a main opening (211) located at said first end (21), to which a deformable element (212) is coupled.

4. Device (1) according to claim 2, characterised
in that it comprises a protection element (23) couplable to said second end (22), and
in that a second opening (231) is afforded on said protection element (23) for the passage of said second end (32) of said elastic element (3).

5. Device (1) according to claim 1, characterised in that said fixing element (4) comprises a first end (41) and a second end (42), both provided with through-holes for enabling passage of said elastic element (3).

6. Device (1) according to claim 5, characterised in that said second end (42) has an appendage (421) able to couple with said deformable element (212) of said introducer element (2).

7. Device (1) according to claim 5, characterised in that a first slot (43) and a second slot (44), diametrically opposite to said first slot (43), are afforded on the external surface of said fixing element (4).

8. Device (1) according to claim 7, characterised in that said first slot (43) is provided with an upper end (431) and a lower end (432), and
in that said second slot (44) is provided with an upper end (441) and a lower end (442).

9. Device (1) according to claim 8, characterised in that it comprises a blocking and unblocking organ (45), able to engage with said first slot (43) and second slot (44), for blocking the elastic element (3) in a predefined position.

10. Device (1) according to claim 9, characterised in that said blocking and unblocking organ (45) comprises a substantially spherical organ (450) connected to a first slider (451) and to a second slider (452), positioned diametrically opposite to one another with respect to said spherical organ (450).
